(19) United States
(12) Patent Application Publication
(10) Pub. No.: US 2024/0341738 A1
(43) Pub. Date: Oct. 17, 2024

(54) Core biopsy tissue sample dislodging onto an adhesives-free strip on an insert fitting into industry-standard cassette for further processing

(71) Applicant: URO-1, Inc, Greensboro, NC (US)

(72) Inventors: Jack Snoke, Winston Salem, NC (US); Philip M. Allred, III, Kernersville, NC (US); Kevin Rackers, Greensboro, NC (US); Ted Belleza, La Selva Beach, CA (US)

(21) Appl. No.: 18/656,006

(22) Filed: May 6, 2024

Related U.S. Application Data
(63) Continuation-in-part of application No. 17/518,442, filed on Nov. 3, 2021, now Pat. No. 11,977,010.
(63) Continuation-in-part of application No. 18/581,296, filed on Feb. 19, 2024.
Application No. 18/581,296 is a continuation-in-part of application No. 18/119,042, filed on Mar. 8, 2023.
Application No. 18/119,042 is a continuation-in-part of application No. 17/082,387, filed on Oct. 28, 2020, now Pat. No. 11,903,569.
(60) Provisional application No. 63/630,443, filed on Feb. 5, 2024.
(60) Provisional application No. 62/955,559, filed on Dec. 31, 2019.

(51) Int. Cl. A61B 10/02; A61B 90/00

(52) U.S. Cl. CPC A61B 10/0275; A61B 90/08; A61B 2090/0811

(57) ABSTRACT

Biopsy tissue sample processing method and system configured to collect a tissue sample in a cradle at a distal portion of a biopsy needle. The cradle has an axially extending floor and two rows of teeth that extend up from sides of the cradle bottom. In each row, the teeth are axially spaced from each other. Dislodging the tissue sample from said cradle involves lightly touching, in a touch-and-go freehand motion of the cradle relative to the strip, an exposed side of the tissue sample in the cradle to a strip that has a surface configured to dislodge the tissue sample from the cradle without a need for an adhesive The cradle teeth protect the tissue sample from radial compression beyond a threshold in said touch-and-go motion and otherwise help maintain sample integrity.

### REFERENCE TO RELATED APPLICATIONS

This patent application is a continuation in-part of each of (1) U.S. patent application Ser. No. 17/518,442 filed Nov. 3, 2021 (scheduled to issue as U.S. Pat. No. 11,977,010 on May 7, 2024) and (2) U.S. patent application Ser. No. 18/581,296 filed Feb. 19, 2024, which claims priority to U.S. Provisional patent application Ser. No. 63/630,443 filed Feb. 5, 2024 and is a continuation in-part of U.S. patent application Ser. No. 18/119,042 filed Mar. 8, 2023, which in turn is a continuation in-part of U.S. patent application Ser. No. 17/082,387 filed Oct. 28, 2020, now U.S. Pat. No. 11,903,569, and claiming priority to U.S. Provisional patent application Ser. No. 62/955,559 filed Dec. 31, 2019.

This application claims priority to and incorporates by reference each of the foregoing provisional and nonprovisional patent applications.

### FIELD

This patent pertains mainly to core biopsy needle sets and to dislodging tissue samples from needles and further processing the samples, including but not limited to samples of prostate tissue.

### BACKGROUND

Core biopsy needle sets have long been used to take samples of tissue. A standard-of-care (SOC) needle set comprises a hollow tubular cannula and a biopsy needle that also is called stylet or core collector and has a pointed tip designed to penetrate tissue. The needle has a notch and is inside the cannula. The cannula has a sharp distal edge. The health practitioner inserts the needle, with the cannula covering the notch, in the patient until the needle tip is near or in target tissue and then releases the spring-loaded needle to shoot distally from the cannula into the tissue to be sampled. When the needle completes its travel, the spring-loaded cannula shoots distally over the needle to cut off and cover a tissue sample that has entered the notch. The practitioner then withdraws the needle set from the patient, pulls the cannula back to expose the tissue sample in the notch, and dislodges the tissue sample from the notch for initial assessment and further processing.

When taking, exposing, and dislodging the tissue sample, it is difficult to maintain the sample's structural integrity. The sample is delicate, typically a mm or so in diameter and a cm or two long, and is susceptible to breaking, stretching, bunching up and otherwise changing from its state when in the tissue from which it was extracted. Such changes in structural integrity can make pathology assessment less reliable and reproducible.

The notch in a standard-of-care (SOC) biopsy needle typically is a cutout in a round needle and has a flat bottom. One traditional way to dislodge tissue from the notch is to swipe the exposed side of the sample on a foam pad or paper. This can require much skill to accomplish without distorting the sample and typically can still introduce sample changes that can interfere with reliable sample assessment. Another traditional way to is swish the needle in a liquid and another is to pick up the sample with tweezers. Both also require much skill and still tends to introduce undesirable distortion. Yet another is proposed in U.S. Pat. No. 10,383,607 and is understood to involve immobilizing an SOC biopsy needle in a jig, with the tissue sample facing up, pressing down on the sample with a levered arm that carries a tissue holder sheet, and then lifting the levered arm. It is believed that when a tissue sample is dislodged on known membrane surfaces, they remain on the membrane during further processing and are wax embedded together with the membrane, and that this makes slicing more difficult and wear out slicing equipment more rapidly.

One objective of this patent specification is to provide core biopsy needles that improve tissue sample integrity and increase tissue sample size compared with using SOC needles to take tissue samples. Another is to provide for dislodging a tissue sample from biopsy needles that is more efficient compared with known SOC methods and is more reliable and better preserves tissue sample integrity. Another is to improve further processing by dislodging the tissue sample on a strip held in an insert that is configured to fit in an industry-standard cassette for washing and to wax-embed only the sample, not the membrane strip, to thereby facilitate slicing the sample for mounting on slides. An example of industry-standard cassettes are the cassettes available from https://shop.leicabiosystems.com/us/histology-consumables/cassettes/pid-biopsy-cassettes

### SUMMARY

This patent specification describes methods and devices for taking and processing core biopsy tissue samples taken with novel core biopsy needles to improve the structural integrity and increase the volume of tissue samples. Both structural integrity and size are important for greater reliability in grossing (initial visual assessment of the tissue samples after they are separated from the biopsy needle) and in further processing and assessment of the samples in a pathology laboratory.

According to some embodiments, dislodging the tissue sample from the new kind of biopsy needle involves lightly touching the exposed side of the tissue sample in the needle to a special strip, in a “touch-and-go” action. The special strip differs from conventional foam pads and tissue sample paper because it is sufficiently charged electrostatically to attract the tissue sample from the biopsy needle and retain it without a need for adhesives. According to other embodiments, the strip has a fibrous surface such as in some cellulose chromatography paper that in effect provides a much larger surface area than a flat surface and tends to grip the tissue sample.

According to some embodiments, preferably a novel core biopsy needle structure helps preserve structural integrity both when taking a tissue sample and when dislodging it onto the strip. Unlike conventional, standard-of-care (SOC) needles with a deep notch for the tissue sample, the novel biopsy needle holds the tissue sample in a cradle with a floor from which two lateral rows of teeth extend up. The teeth of each row are spaced from each other axially to leave spaces between adjacent teeth. This cradle structure helps collect larger samples compared with SOC needles. The teeth help keep the sample from pressure distortion and the spaces between the teeth help keep the tissue sample from persisting axial distortion.

Compared with using a standard-of-care (SOC) biopsy needle with a deep notch and no teeth and SOC swiping or dragging, while bending the notch, onto a foam pad or swishing in a liquid to dislodge a tissue sample from SOC needles, the new method and devices disclosed in this patent specification achieve (a) less tissue sample fragmentation, (b) less tissue sample stretching during dislodging from a biopsy needle, (c) tissue samples that are straighter and flatter during fixation, (d) tissue samples that better replicate the tissue when in the environment from which it was taken, (e) faster and more reliable grossing and tissue quality evaluation, (f) easier wax embedding with tissue samples that lay flat, and (g) easier and more reliable slicing in a microtome for better slice image quality/diagnosis.

According to some embodiments, the strips are held in inserts configured to fit in industry-standard cassettes for washing. The washed tissue samples preferably are wax-embedded without the strips.

As described in the initially presented claims but subject to amendments thereof in prosecuting this patent application, according to some embodiments a core biopsy tissue sample processing comprises: collecting a tissue sample that has an exposed side and a portion that is in a cradle at a distal portion of a core biopsy needle, which cradle comprises an axially extending floor and two rows of teeth that extend up from sides of the floor such that there are axial spaces between axially adjacent teeth of each row; and dislodging the tissue sample from said cradle by lightly touching, in a touch-and-go freehand motion of the cradle relative to the strip, the exposed side of the tissue sample to a strip of a material configured to dislodge the tissue sample from the cradle onto the strip without a need for an adhesive; wherein said teeth keep the tissue sample from radial compression beyond a selected threshold in said touch-and-go motion.

According to some embodiments, the processing includes one or more of: (a) the strip onto which the tissue sample is dislodged comprises electrostatically charged Nylon 66; (b) the strip onto which the tissue sample is dislodged in said touch-and-go motion comprises fibrous cellulose paper; (c) the strip onto which the tissue sample is dislodged in said touch-and-go motion comprises Cellulose Chromatography Paper; (d) placing the strip into a strip platform of an insert with side channels open to each other before dislodging the tissue sample onto the strip in said touch-and-go motion; (e) sliding the strip into a strip platform of an insert with side channels open to each other before dislodging the tissue sample onto the strip in said touch-and-go motion, wherein said open side channels include projections configured to engage said strip against undesired sliding; (f) placing said strip into a platform of an insert, wherein said insert has side channels open toward each other into which sides of said strip fit and a needle icon structure indicating how to orient said tissue sample relative to axial ends of the strip to thereby indicate an anatomical orientation of the tissue sample and with a scale to visually assess length of the sample on the strip; (g) supporting said strip, before said tissue sample is dislodged thereon, in an insert; placing said insert, with the strip in the insert and the tissue sample on the strip, in a jar filled with sufficient fixative to enable the tissue sample to be completely covered with the fixative, wherein said insert is configured to allow free flow of said fixative to said tissue sample; and transporting the jar with the insert therein to a pathology laboratory, extracting the insert from the jar, grossing the sample while on the insert, placing the insert with the tissue sample therein in an industry-standard cassette, wherein the insert is dimensioned to fit freely but snugly in the cassette, placing a patch of soft and permeable material over the sample, closing the cassette and washing the sample while in the cassette, opening the cassette and extracting the tissue sample therefrom, placing the extracted tissue sample alone in wax bath and solidifying the wax with the tissue sample therein for slicing and further processing; and (h) supporting said strip, before the tissue sample is dislodged thereon, in an insert, and supporting additional strips in additional inserts before respective tissue samples are dislodged therein, to thereby provide a plurality of inserts each placed in an industry-standard cassette labeled with patient data and each supporting a respective strip with a respective tissue sample thereon, in a single jar filled with enough fixative to enable the tissue samples on the plural inserts to be completely covered with the fixative, wherein said inserts are configured to enable free flow of said fixative to the tissue samples therein; and transporting the jar with the inserts therein to a remote pathology laboratory, extracting the inserts from the jar, grossing the tissue samples while on the respective inserts, placing the inserts with the respective tissue samples therein in an industry-standard cassette labeled with patient data, wherein each insert is dimensioned to fit freely but snugly in a respective cassette, placing a patch of soft and permeable material over each sample, closing the cassettes and washing the samples while in the cassettes, opening the cassettes and extracting the tissue samples therefrom, placing the extracted tissue samples alone in one or more wax baths and solidifying the wax with the tissue samples therein for slicing and further processing.

According to some embodiments, a core biopsy tissue sample processing system comprises: a core biopsy needle that has a distal portion with a cradle comprising an axially extending floor and two rows of teeth that extend up from sides of the floor, with the teeth of each row spaced axially from each by inter-tooth axial spaces, wherein said cradle is configured to hold a tissue sample that has an exposed side; and an insert comprising a strip platform, a strip of a material thereon that is configured to dislodge a tissue sample from said cradle by lightly touching the exposed side of the tissue sample in a touch-and-go freehand motion of the cradle relative to the strip, without a need for an adhesive; wherein said teeth are configured to keep the tissue sample from radial compression beyond a selected threshold in said touch-and-go motion of the cradle relative to the strip.

According to some embodiments, the system includes one or more of: (a) said strip comprises Nylon 66 that is electrostatically positively charged sufficiently to dislodge said tissue sample without adhesives in said touch-and-go motion; (b) said strip comprises fibrous cellulose paper that is configured to dislodge said tissue sample without adhesives in said touch-and-go motion; (c) said strip comprises Cellulose Chromatography Paper that is configured to dislodge said tissue sample without adhesives in said touch-and-go motion; (d) said strip comprises a polyamide that is electrostatically positively charged sufficiently to dislodge said tissue sample from said cradle by said touch-and-go motion; (e) said insert includes channels at sides of said strip platform that are open to each other and into which sides of said strip slide; (f) said insert includes surface features configured to retain said strip in place over said platform; and (g) said insert is configured to fit in an industry-standard biopsy sample cassette that has an interior opening with sides 30-35 mm by 25-30 mm and 4-6 mm depth.

According to some embodiments, a core biopsy tissue sample processing system comprises: a core biopsy needle that has a distal portion comprising an axially extending reduced-diameter portion configured to contain a tissue sample that has an exposed side; and an insert comprising a strip platform, a strip of a material thereon that has a surface configured to dislodge a tissue sample from said reduced-diameter portion by lightly touching the exposed side of the tissue sample in a touch-and-go freehand motion of the biopsy needle relative to the strip, without a need for an adhesive.

According to some embodiments, the system described in the immediately preceding paragraph includes one or more of: the strip comprises Nylon 66 that is electrostatically charged positively sufficiently to dislodge said negatively tissue sample without adhesives in said touch-and-go motion; (b) said strip comprises Cellulose Chromatography Paper that is configured to dislodge said tissue sample without adhesives in said touch-and-go motion; and (c) said insert is dimensioned to fit in an industry-standard biopsy sample cassette that has an interior opening with sides 30-35 mm by 25-30 mm and 4-6 mm depth.

### BRIEF DESCRIPTION OF THE DRAWINGS

FIG. 1 is a perspective view of insert holding a strip with a tissue sample thereon, wherein the strip has a surface configured to help dislodge a tissue sample from a biopsy needle without a need for adhesives, according to some embodiments.

FIG. 2 is a perspective view of a biopsy needle from which a tissue sample can be dislodged onto a strip, according to some embodiments.

FIG. 3 is a side view of a distal portion of a biopsy needle and of a distal portion of a cannula surrounding the needle, according to some embodiments.

FIG. 4 is a perspective view of the insert without a strip or a tissue sample therein, according to some embodiments.

FIG. 5 is a top view of a cassette, according to some embodiments.

FIG. 6 is a section of the insert of FIG. 5 taken at A-A and FIG. 6A is an elevation viewed from, the lower side of FIG. 5, according to some embodiments.

FIG. 7 is a section that is otherwise like FIG. 6 but adds a section of a strip and of a cradle of a biopsy needle with a tissue sample therein being dislodged on the strip.

FIGS. 8A-8F are sectional views illustrating stages of dislodging a tissue sample onto a strip from a core biopsy needle cradle that has teeth extending from the cradle floor.

FIGS. 9A-9C are sectional views illustrating dislodging a tissue sample from a standard-of-care prior at core biopsy needle.

FIG. 10 is a perspective view of a tray with small jars each for holding an individual insert with a tissue sample thereon covered in fixative, according to some embodiments.

FIG. 11 is a perspective view of a large jar for a plurality of industry-standard cassettes each containing a respective insert with a tissue sample on a strip, covered in fixative, according to some embodiments.

FIG. 12 is a perspective view of an industry-standard cassette for biopsy tissue, according to some embodiments.

FIG. 13 is a perspective view of an industry-standard foam pad for use in the cassette, according to some embodiments.

FIG. 14 is a flow chart of steps in processing core biopsy tissue samples in case an external pathology laboratory is used, according to some embodiments.

FIG. 15 is a flow chart of steps in processing core biopsy tissue samples in case an in-house pathology laboratory is used, according to some embodiments.

### DETAILED DESCRIPTION OF EXAMPLES

A detailed description of examples of preferred embodiments is provided below. While several embodiments are described, the new subject matter described in this patent specification is not limited to any one embodiment or combination of embodiments described herein, but instead encompasses numerous alternatives, modifications, and equivalents. In addition, while numerous specific details are set forth in the following description to provide a thorough understanding, some embodiments can be practiced without some or all these details. Moreover, for the purpose of clarity, certain technical material that is known in the related art has not been described in detail in order to avoid unnecessarily obscuring the new subject matter described herein. It should be clear that individual features of one or several of the specific embodiments described herein can be used in combination with features of other described embodiments or with other features. Like reference numbers and designations in the various drawings indicate like elements.

Like reference numbers and designations in the various drawings indicate like elements. Further, the reference numbers of components that are like in structure and function have the same second and third digits. For conciseness, components that bear the same reference numbers of the same last two digits of reference numbers are described only in connection with the Figure that first refers to them and the description is not repeated in connection with subsequently discussed Figures.

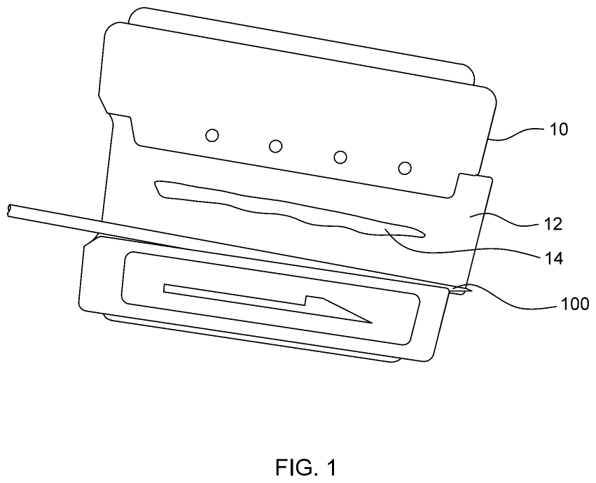

FIG. 1 is a perspective view of insert 10 holding strip 12 on which a tissue sample 14 from core biopsy needle 100 is dislodged without a need for adhesives. Strip 12 can be a material that is sufficiently electrostatically charged to help dislodge tissue sample 14 or can be a material that has a fibrous surface that tends to grip tissue sample 145 to help dislodge it from needle 100.

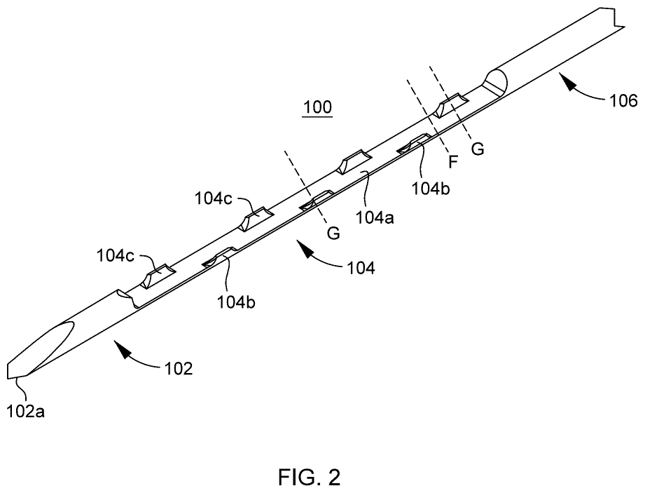

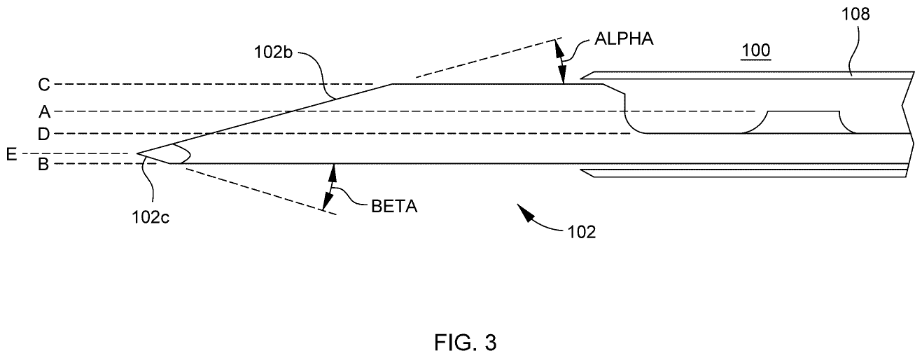

FIGS. 2 and 3 illustrate core biopsy needle 100 in more detail and are taken from said parent application Ser. No. 18/581,296.

Referring to FIGS. 2 and 3, biopsy needle 100 comprises tip 102, cradle 104 proximal to the tip, and shaft 106 proximal to the cradle. Central axis A is at the center of a cross-section through shaft 106. The bottom extent of needle 100 is at level B and the top extent of needle 100 is at level C. Shaft 106 has a portion (not shown) that extends further to the right and has a proximal end configured for attachment, fixed or removable, to a biopsy gun (not shown). Core biopsy needle 100 typically is a part of a core biopsy needle set that further includes a cutting cannula 108. A distal portion of cannula 108 is seen in FIG. 3. Cutting cannula 108 has a sharp distal end 108a and is configured to slide distally and proximally while surrounding biopsy needle 100. Cannula 108 has a proximal portion (not shown) that extends further to the right and also is configured for attachment to a biopsy gun. It should be clear that positional terms such as upper, lower, above, below, left, right, etc. refer to the orientation of biopsy needle 100 illustrated in the figures and that different orientations of needle 100 and insert 10 relative to an observer would require adapting these positional terms accordingly.

Cradle 104 comprises an upwardly facing floor 104a, a left row of teeth 104b extending up from a left side of floor 104a, and a right row of teeth 104c extending up from a right side of floor 104a. Floor 104a is at a level D that is below central axis A. In some embodiments, teeth 104b can extend up to, or approximately up to, the level of central axis A, i.e., to or approximately to half the diameter of biopsy needle 100 in cross-section of shaft 106, as do teeth 104c. Teeth 104b, in the left row of teeth, are spaced axially from each other. Teeth 104c, in the right row of teeth, also are spaced axially from each other. For example, the teeth in a row can be spaced axially as illustrated in FIG. 2, scaled to the dimensions of a specific biopsy needle. In the example of FIG. 2, each tooth is spaced from an adjacent tooth in the same row by 2-3 times the axial length of a tooth at the level of cradle floor 104a. In this example, the most distal tooth is spaced from tip 102 by at least the axial length of one tooth and the most proximal tooth is spaced from shaft 106 by at least the axial length of a tooth. As an example, for a cradle 104 that is 20 mm in axial length, one row can have 3 teeth and the other 4 teeth.

Preferably, the teeth of the left and right rows are staggered such that some cross-sections through cradle 104, such as sections G, include a single tooth such as 104b or 104c, but some sections such as F do not include any such teeth. For example, cross-section F includes no teeth and thus no structure above cradle floor 104a.

FIG. 3 illustrates a core biopsy needle 100 that in some embodiments has a unique shape of the distal portion of tip 102 in that distal cutting edge 102a is raised above the level B of the bottom extent of biopsy needle 100 and this cutting edge 102a is elongated in a direction parallel to or generally parallel to cradle floor 104a (which can be flat or curved such as somewhat concave or convex), as described in more detail in said parent application Ser. No. 18/581,296. Tip 102 terminates distally in a sharp distal cutting edge 102a formed at the convergence of upper and lower facets 102b and 102c. Cutting edge 102a is at a level E that is up from the level B of the bottom extent of needle 100 but is below the level D of floor 104a cradle 104. Level E of cutting edge 102a preferably is up from level B by about a third of the distance between levels B and D, but this can vary somewhat. In a specific example, the distance is 15 thousands of an inch, or is that distance plus or minus 5 thousands of an inch. In other embodiments, needle 100 can have a different tip, for example a tip coming to a point at the level of plane B.

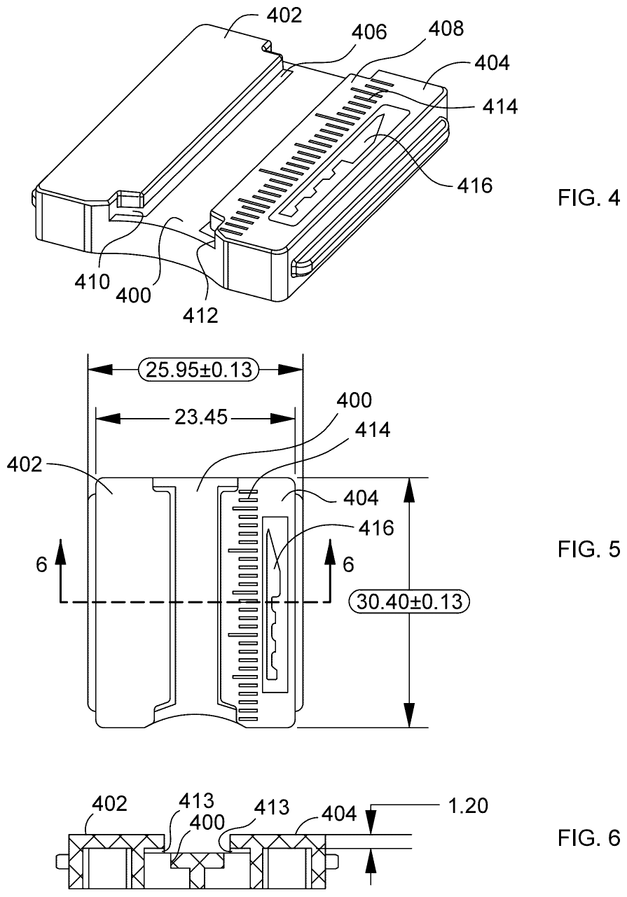

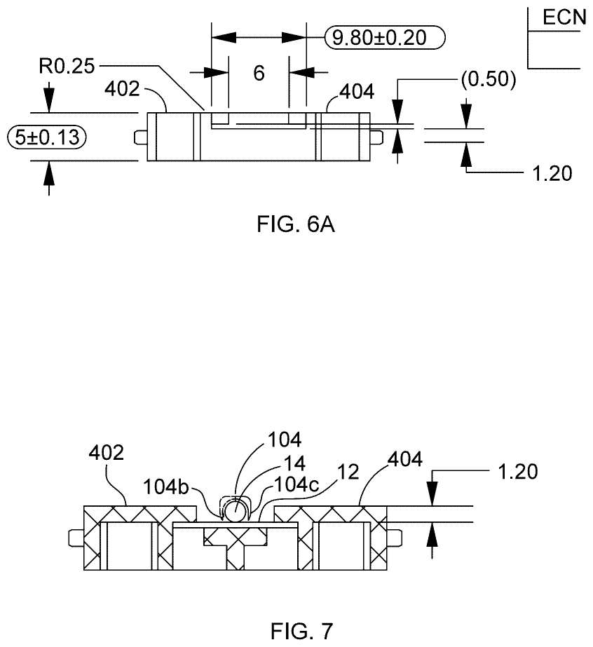

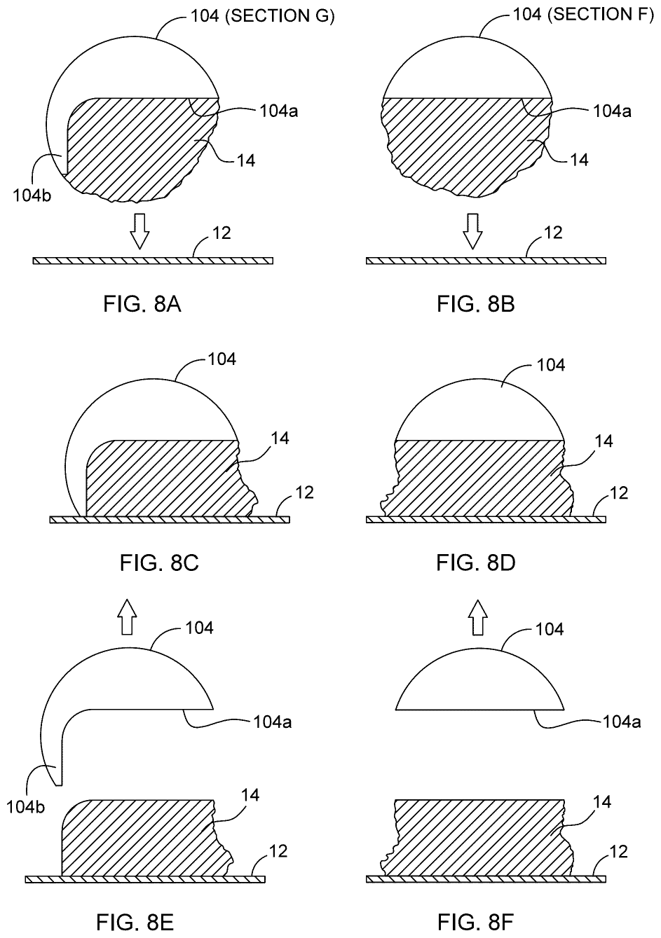

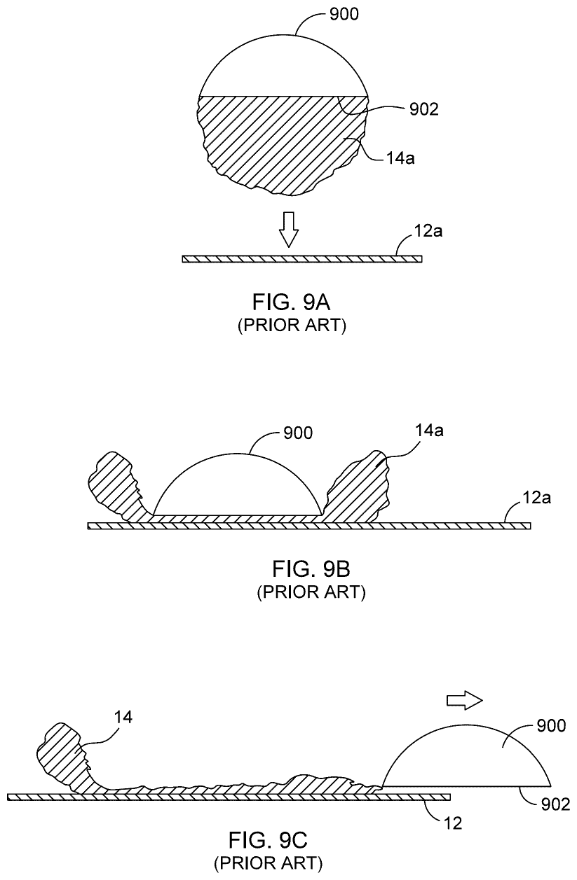

FIG. 4 is a perspective view of insert 10 before loading strip 12 therein and before dislodging tissue sample 14 to strip 12, FIG. 5 is a top view of the insert, FIG. 6 is a section taken at A-A of FIG. 5 and FIG. 6A is an elevational view, and FIG. 7 is otherwise like FIG. 6 but adds a sectional view of strip 12 and of cradle 104 of biopsy needle 100 with a tissue sample 14 therein being dislodged to strip 12. FIGS. 8A-8F illustrate steps in dislodging tissue sample 14 on strip 12, and FIGS. 9A-9C illustrate excessive compression and distortion that can result when a tissue sample 14 is dislodged from an SOC needle.

Referring to FIGS. 4-7, insert 10 comprises a central plate 400 that faces up and is between and below upper plates 402 and 404, which have extensions 406 and 408 that overhang plate 400 and extend toward each other to form inwardly open axial channels 410 and 412. As best seen in FIG. 7, strip 12 rests on plate 400 such that its lateral sides slide, preferably removably, into channels 410 and 412. Preferably, insert 10 is made of molded hard plastic material. Strip 12 typically is cut from sheet or roll of flat stock. Strip 12 can be slid over plate 400 at the time of manufacture of insert 10 or at the time insert 10 is to be used to dislodge a biopsy sample to strip 12, or at some other time. Preferably, strip 12 faces surfaces in channels 410, 412 that are sufficiently barbed or roughened to allow strip 12 to be slid in place but to keep it from sliding out. Barbs or other surface roughening 413 is seen in FIG. 6.

Strip 12 is made of special material that enables tissue sample 14 to be dislodged on strip 12 by a light, touch-and-go motion without adhesives on strip 12. One example is a strip that is sufficiently charged electrostatically to dislodge a tissue sample 14 held in cradle 104 using a touch-and-go motion described further below. An example of such material for strip 12 is electrostatically positively charged Nylon 66 available for example from Roche Diagnostics GmbH of Mannheim, Germany under the designation Nylon Membranes, positively charged. While the discharge mechanism is complex and may not be entirely understood, it is believed that the positive electrostatic charge of strip 12 interacts with the typically negatively charged tissue sample 14 to help dislodge the tissue sample with only a light touch-and-go motion of needle 100 relative to strip 12. Another example for the material of strip 12 is Cytiva Chromatography Paper Ct. No. 3030-335 (see https://www.cytivalifesciences.com/en/us/shop/protein-analysis/blotting-and-detection/blotting-papers/grade-3 mm-chr-cellulose-chromatography-papers-p-00921). This material has a fibrous surface with a side from which fibers extend up to provide an effective, total surface area of the many fibers that is sufficiently large compared with a smooth surface to tend to grip tissue sample 14 sufficiently to dislodge it without a need for adhesives with only light touch-and-go motion of needle 100 relative to strip 12.

Visible surfaces of upper plates 402 and 404 can have indicia structures to assist in processing tissue samples 14. For example, a scale 414 can be formed on upper plate 404, close to its inboard edge, with marks indicative of distance, for example 1 mm marks, to assist in initial grossing (visually assessing the length and other characteristics of a tissue sample 14). In addition, a large arrow 416 on upper plate 404 can indicate toward which axial end of plate 400 to orient the distal end of biopsy needle 100 and thus preserve an indication of the orientation of tissue sample 14 in the tissue from which it was extracted. Additional markings can be provided on the same upper plate or on upper plate 402.

Referring to FIG. 7, dislodging tissue sample 14 onto strip 12 only requires a light touch of the exposed side of sample 14 that is in cradle 104 of biopsy needle 100, to strip 12 in a touch-and-go motion of needle 100 held freehand. Typically, a practitioner holds the biopsy gun to which biopsy needle 100 is attached and moves cradle 104 freehand to strip 12, with the exposed side of tissue sample 14 facing down, and lightly touches tissue sample 14 to strip 12, at a location about halfway between upper plates 402, 404. The practitioner may initially touch an axial end of tissue sample 14 near an axial end of strip 12 and can then move biopsy needle 100 to roughly parallel to strip 12 to thereby contact more or all of tissue sample 14 to strip 12, or may start with orienting biopsy needle 100 to initially touch more or most or all of tissue sample 14 to strip 12. According to some embodiments, strip 12 is sufficiently electrostatically charged to attract tissue sample 12 that it contacts or nearly contacts to thereby enable the practitioner to gently lift biopsy needle 100 from strip 12 while leaving tissue sample 14 on strip 12. No adhesives or special liquids need be used to accomplish this. The practitioner can dislodge tissue sample 14 onto strip 12 by initially lifting up one axial end or cradle 104 and continuing until the entire tissue sample 14 remains on strip 12, or may additionally or instead rock biopsy needle 100 side-to-side or rotate is gently or otherwise assist in the transfer of the tissue sample, or may lift cradle 104 straight up. According to other embodiments, when a fibrous material strip 12 is used that has a side from which fibers extend up to provide a sufficiently large effective surface area to grip a tissue sample, the practitioner uses a like touch-and-go motion to dislodge tissue sample 14 on strip 12.

Importantly, teeth 104b and 104c assist in maintaining the structural integrity of tissue sample 14 in the process of dislodging it onto strip 12. First, teeth 104b and 104c limit pressure that can be exerted on tissue sample 14 because if teeth 104b and 104c contact strip 12, tissue sample 14 cannot be pressed any more. This is unlike using a conventional biopsy needle that has a simple deep notch, with no teeth, which can allow much more compression and thus structural distortion of a tissue sample in similar settings. Second, teeth 104b are axially spaced from each other, and so are teeth 104c, and the axial spaces between adjacent teeth allow tissue to resiliently expand sideways therein and spring back upon lifting cradle 104 from strip 12 and the tissue sample that remains on the strip. Also, the axial spaced between teeth and the staggering of teeth help ease separation of the tissue sample from cradle 104 onto strip 12.

FIGS. 8A-F show cross-sections though cradle 104 at different stages of dislodging tissue sample 14 on strip 12. FIGS. 8A, C, and E show a section F that includes tooth 104B (see FIG. 2) and FIGS. 8B, D, and E show a section through trough section F that does not include any teeth. A section G that includes a tooth 10C is not shows as it is a mirror image of the section in FIGS. 8A, C, and E. FIGS. 8A and 8B show positions of cradle 104 and tissue sample 14 just before the exposed bottom side of sample 14 touches strip 12. For clarity, these figures do not show insert 10 but it should be clear that strip 12 is in position on insert 10 like that shown in FIG. 1. FIGS. 8C and 8D show positions when dislodging sample 14. FIGS. 8E and 8F show tissue sample 14 dislodged on strip 12 and cradle 104 lifted up from strip 12. Note FIG. 8C shows that tooth 104B prevents excessive compression of tissue sample 14. Teeth 104B, on the other side of floor 104a of cradle 104, keep tissue sample 14 from excessive compression in the same manner. Also note that FIG. 8C shows a slight protrusion of tissue sample 14 to the right, in a space between two adjacent teeth 104c (which are not seen in FIG. 8C). A like protrusion would occur between two adjacent teeth 104b in the other row of teeth. FIG. 8D shows a like protrusion to right and left as there are no teeth in the section that FIG. 8D shows. These protrusions may or may not occur, depending on downward pressure on tissue sample 14 and depending on the nature of the sample and on how it was obtained. This process can help maintain structural integrity of the tissue sample as they allow some flexing and recovery of the tissue in the process of obtaining the sample and the process of dislodging it on strip 12. Finally, FIGS. 8E and 8F show sample 14 resting on strip 12 after cradle 104 has been lifted from the sample. The side protrusions, if any, may remain or the tissue may spring back to a roughly round shape.

FIGS. 9A, 9B, and 9C show dislodging a tissue sample 14 using a standard of care core biopsy needle 900 that has a simple deep notch 902, with no teeth, holding and dislodging a tissue sample 14a. FIG. 9A shows tissue sample 14a just above a strip or membrane 12a, which can be a conventional tissue sample foam or paper. The practitioner typically presses notch 902 down on membrane 12a and can compress it to the extent shown in FIG. 9B as there are no teeth to limit compression. The practitioner may then swipe needle 900 sideways to leave sample 14a on membrane 12a. The sample may be severely distorted. Less pressure on the sample can mean less distortion but it also may mean failure to dislodge the sample and a repeat of the dislodge attempt. FIGS. 9A-C may show exaggerated distortion but tests have shown that obtaining a tissue sample and dislodging it using a core biopsy needle of the kind shown in FIGS. 2 and 3 provide higher quality samples. See for example the Declarations of Drs. Proctor and Bonham and the Posters attached thereto, submitted in said parent application Ser. No. 17/082,387.

The processing of biopsy tissue samples can proceed further. A first example of such processing is when tissue samples 14 are collected in a clinic or office that does not have an in-house pathology laboratory but sends the samples to a remote lab. A second example is when the clinic or office that carries out the core biopsy to obtain the samples has an in-house pathology laboratory.

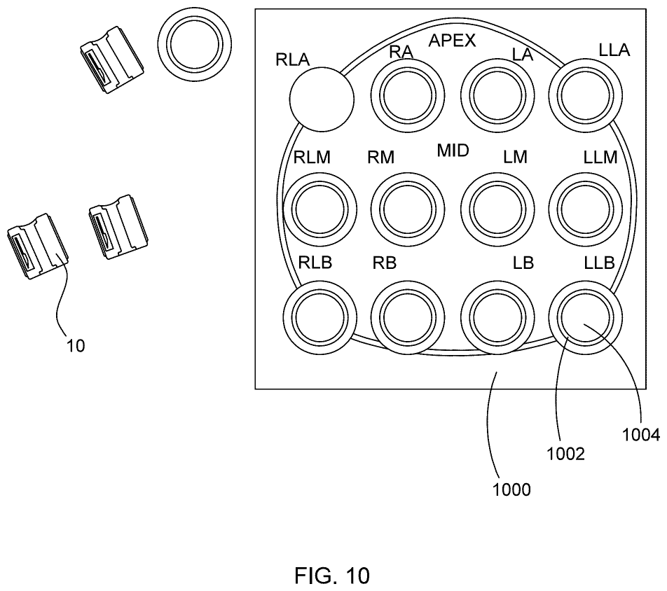

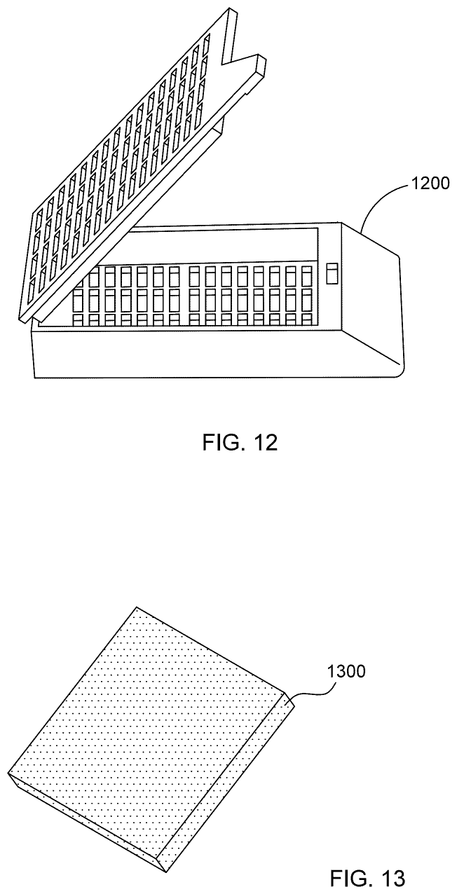

In the first example, the practitioner collects a tissue sample 14 with needle 100 and the practitioner or an assistant dislodges the sample on strip 12 in insert 10 as described above, using a light touch-and-go motion. The practitioner or assistant places insert 10, with strip 12 in place and tissue sample 14 on strip 12, in a vertical position into a small jar, for example a 20 ml jar, filled with enough fixative (formalin) to enable the vertical oriented tissue sample to be completely covered with the formalin fixative. Tissue samples typically are transported to a pathology lab to be processed within 24-48 hours of taking them. Adequate formalin flow/contact is important to complete the fixing such that insert 10 remains immersed in the jar. FIG. 10 shows an example of a tray 1000 and 16 jars 1002 each containing an insert 10 with a respective tissue sample 14 on a strip 12 therein. Stickers 1004 may be placed on jars 1002 to identify the respective samples and/or provide other information. Tray 1000 is shipped to a pathology laboratory, where a practitioner opens the tray, takes each jar 1002 in turn, opens the jar and extracts insert 10 from the jar, with sample 14 still on strip 12 in the insert. The first step at the path lab is grossing the sample, measuring length with sample 14 on strip 12 and using the mm scale 414 (FIGS. 4 and 5) on insert 10. The anatomical orientation of sample 14 is indicated by arrow 416, which is a needle icon. The practitioner may dye one axial end of sample 14 with tissue marking dye to note and preserve information about the position of the sample in relation to the gland or organ that the sample is taken from. The second step is to place insert 10, still with sample 14 on strip 12, into a cassette 1200 (FIG. 12) that is labeled with patient information and with a foam pad 1300 (FIG. 13—standard biopsy product) placed on of top of insert 10 to prevent tissue disruption. Cassette 1200 may be an industry-standard cassette example identified herein. The practitioner then closes the cassette lid. The third step is washing in a processor. The labeled cassette 1200 with sample on the insert 10 in the cassette goes through several washing cycles while remaining closed. In the fourth step, after the washing is completed, cassette 1200 is opened, foam pad 1300 is removed, and sample 14 is removed from strip 12 and placed in a mold that is filled with wax and chilled to set the wax. Note that only tissue sample 14, without strip 12, is placed in the wax. The fifth step is slicing the wax block in a microtome. The thin slices are floated in a water bath and then picked up and placed on a microscope slide. The prepared slides are then ready for diagnostic analysis by a pathologist.

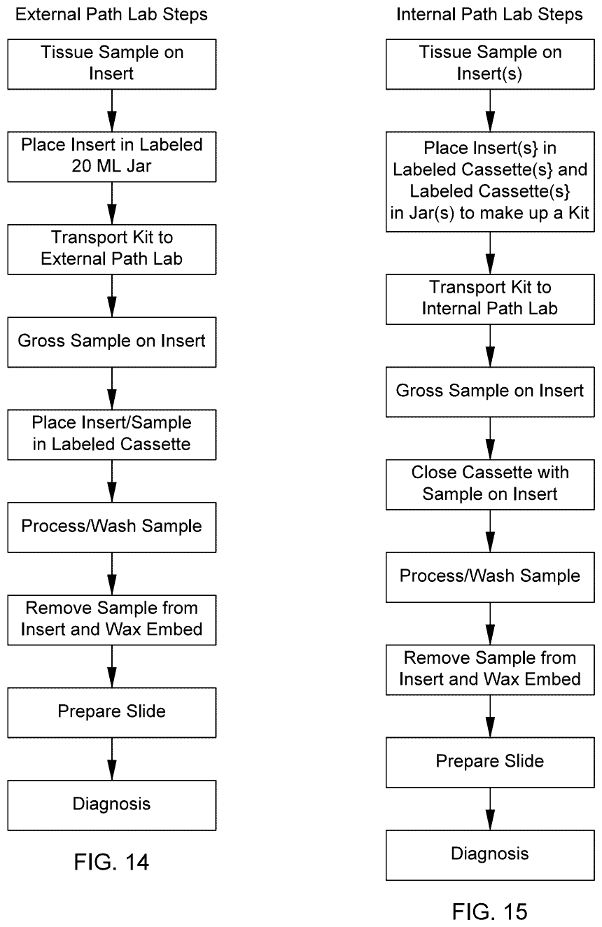

FIG. 14 is a flowchart of steps in a process that starts with taking a tissue sample 12 with core biopsy needle 100 and dislodging it in an insert 10 and ends with diagnosis of the tissue sample in an external pathology laboratory that is remote from the facility such as a clinic, medical office, or a hospital in which the tissue sample was obtained from a patient.

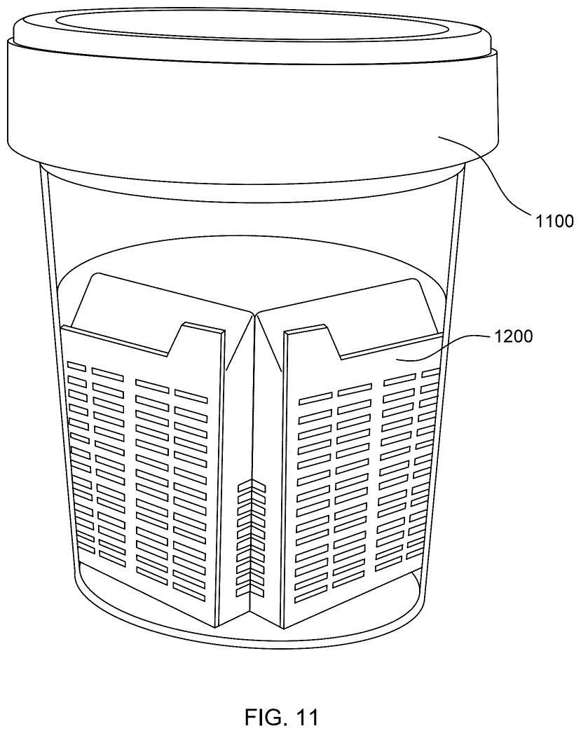

In the second example, when there is an in-house pathology lab in the clinic or office or hospital where the tissue samples are taken, tissue sample 14 also is dislodged on strip 12 as described above. Insert 10, with strip 12 and sample 14 on strip 12 therein, is placed in a cassette 1200, for example the industry-standard cassette identified herein, preferably labeled with patient information. A foam pad 1300 (FIG. 13) might be placed over the sample to keep it in place. Several, for example six such inserts 10, each in a respective cassette 1200, are placed in a larger jar 1100 (FIG. 11), for example a 120 ml jar, where they are immersed in fixative. A large jar 100 can be used to enclose all the required cassettes 1200, or two or more jars 1100 can be used. Adequate formalin flow/contact is important to complete the fixing. The kit of one or more larger jars 1100 is carried to the in-house processing lab to be processed within 24-48 hours. The first step at the in-house path lab is to open each cassette 1200 to expose sample 14 therein, and gross the sample, measuring length with the sample on the strip and using the mm scale 414 printed on the insert while in the cassette. The anatomical orientation is indicated by needle icon 416 and one axial end of sample 14 can be dyed with tissue marking dye to note the position in which the sample was in relation to the gland or organ that the sample is taken from. The second step is to place a foam pad 1300 on top of sample 14 in insert 10 in each cassette 1200 to prevent any tissue disruption, place insert 10 in an industry-standard cassette 1200 labeled with patient information and close the cassette lid. The third step is washing the cassette in a processor. The labeled cassette with sample 14 on strip 12 in insert 10 inside goes through several washing cycles while remaining closed. The fourth step, after the washing is completed, is to open the cassette, remove the foam pad and place sample 14, without strip 12, in a mold that is filled with wax and chilled to set the wax. The fifth step is slicing the wax block in a microtome. The thin slices are floated in a water bath and then picked up and placed on a microscope slide. The prepared slides are then ready for diagnostic analysis by a pathologist.

FIG. 15 is a flowchart of steps in a process that starts with taking a tissue sample 12 with core biopsy needle 100 and dislodging it in an insert 10 and ends with diagnosis of the tissue sample in an external pathology laboratory that is in-house, i.e., in the same such as a clinic, medical office, or a hospital in which the tissue sample was obtained from a patient.

An important aspect of the processes illustrated in FIGS. 14 and 15 is that preferably insert 10 is dimensioned to fit freely but snugly in an industry-standard cassette such as the cassette example identified above. FIG. 12 illustrates a typical industry-standard cassette and FIG. 13 illustrates an industry-standard foam pad that can be used to place over insert 10 to keep tissue sample 14 in place after the cassette is closed. For example, an insert 10 shown in FIG. 5 can be roughly 26×30.5 mm, and its thickness can be less than 4-6 mm. One example of an industry-standard foam pad as shown in FIG. 12 is 30.2 mm×25.4 mm×2 mm in size, with an interior opening, in which insert 10 fits, with sides 30-35 mm by 25-30 mm and 4-6 mm depth. Making inserts 10 fit in industry-standard cassettes has been found uniquely advantageous because pathology labs often have such cassettes and foam pads in stock and thus can avoid having to stock different size cassettes.

Although the foregoing has been described in some detail for purposes of clarity, it will be apparent that certain changes and modifications may be made without departing from the principles thereof. There can be many alternative ways of implementing both the processes and apparatuses described herein. Accordingly, the present embodiments are to be considered as illustrative and not restrictive, and the body of work described herein is not to be limited to the details given herein, which may be modified within the scope and equivalents of the appended claims.

1. Core biopsy tissue sample processing comprising:
collecting a tissue sample that has an exposed side and a portion that is in a cradle at a distal portion of a core biopsy needle, which cradle comprises an axially extending floor and two rows of teeth that extend up only from sides of the floor and are axially spaced from each other such that there are axial spaces between axially adjacent teeth of each row; and
dislodging the tissue sample from said cradle by lightly touching, in a touch-and-go freehand motion of the cradle relative to the strip, the exposed side of the tissue sample to a strip of a material configured to dislodge the tissue sample from the cradle onto the strip without a need for an adhesive;
wherein said teeth keep the tissue sample from radial compression beyond a selected threshold in said touch-and-go motion.

2. The core biopsy tissue sample processing of claim 1, in which the strip onto which the tissue sample is dislodged comprises electrostatically charged Nylon 66.

3. The core biopsy tissue sample processing of claim 1, in which the strip onto which the tissue sample is dislodged in said touch-and-go motion comprises fibrous cellulose paper.

4. The core biopsy tissue sample processing of claim 1, in which the strip onto which the tissue sample is dislodged in said touch-and-go motion comprises Cellulose Chromatography Paper.

5. The core biopsy tissue sample processing of claim 1, further comprising placing the strip into a strip platform of an insert with side channels open to each other before dislodging the tissue sample onto the strip in said touch-and-go motion.

6. The core biopsy tissue sample processing of claim 1, further comprising sliding the strip into a strip platform of an insert with side channels open to each other before dislodging the tissue sample onto the strip in said touch-and-go motion, wherein said open side channels include projections configured to engage said strip against undesired sliding.

7. The core biopsy tissue sample processing of claim 1, further comprising placing said strip into a platform of an insert, wherein said insert has side channels open toward each other into which sides of said strip fit and a needle icon structure indicating how to orient said tissue sample relative to axial ends of the strip to thereby indicate an anatomical orientation of the tissue sample and with a scale to visually assess length of the sample on the strip.

8. The core biopsy tissue sample processing of claim 1, further comprising:
supporting said strip, before said tissue sample is dislodged thereon, in an insert;
placing said insert, with the strip in the insert and the tissue sample on the strip, in a jar filled with sufficient fixative to enable the tissue sample to be completely covered with the fixative, wherein said insert is configured to allow free flow of said fixative to said tissue sample; and
transporting the jar with the insert therein to a pathology laboratory, extracting the insert from the jar, grossing the sample while on the insert, placing the insert with the tissue sample therein in an industry-standard cassette, wherein the insert is dimensioned to fit freely but snugly in the cassette, placing a patch of soft and permeable material over the sample, closing the cassette and washing the sample while in the cassette, opening the cassette and extracting the tissue sample therefrom, placing the extracted tissue sample alone in wax bath and solidifying the wax with the tissue sample therein for slicing and further processing.

9. The core biopsy tissue sample processing of claim 1, further comprising:
supporting said strip, before the tissue sample is dislodged thereon, in an insert, and supporting additional strips in additional inserts before respective tissue samples are dislodged therein, to thereby provide a plurality of inserts each placed in an industry-standard cassette labeled with patient data and each supporting a respective strip with a respective tissue sample thereon, in a single jar filled with enough fixative to enable the tissue samples on the plural inserts to be completely covered with the fixative, wherein said inserts are configured to enable free flow of said fixative to the tissue samples therein; and
transporting the jar with the inserts therein to a remote pathology laboratory, extracting the inserts from the jar, grossing the tissue samples while on the respective inserts, placing the inserts with the respective tissue samples therein in an industry-standard cassette labeled with patient data, wherein each insert is dimensioned to fit freely but snugly in a respective cassette, placing a patch of soft and permeable material over each sample, closing the cassettes and washing the samples while in the cassettes, opening the cassettes and extracting the tissue samples therefrom, placing the extracted tissue samples alone in one or more wax baths and solidifying the wax with the tissue samples therein for slicing and further processing.

10. A core biopsy tissue sample processing system comprising:
a core biopsy needle that has a distal portion with a cradle comprising an axially extending floor and two rows of teeth that extend up from only sides of the floor, with the teeth of each row spaced axially from each by inter-tooth axial spaces, wherein said cradle is configured to hold a tissue sample that has an exposed side; and
an insert comprising a strip platform, a strip of a material thereon that is configured to dislodge a tissue sample from said cradle by lightly touching the exposed side of the tissue sample in a touch-and-go freehand motion of the cradle relative to the strip, without a need for an adhesive;
wherein said teeth are configured to keep the tissue sample from radial compression beyond a selected threshold in said touch-and-go motion of the cradle relative to the strip.

11. The core biopsy tissue sample processing system of claim 10, in which said strip comprises Nylon 66 that is electrostatically positively charged sufficiently to dislodge said tissue sample without adhesives in said touch-and-go motion.

12. The core biopsy tissue sample processing system of claim 10, in which said strip comprises fibrous cellulose paper that is configured to dislodge said tissue sample without adhesives in said touch-and-go motion.

13. The core biopsy tissue sample processing system of claim 10, in which said strip comprises Cellulose Chromatography Paper that is configured to dislodge said tissue sample without adhesives in said touch-and-go motion.

14. The core biopsy tissue sample processing system of claim 11, in which said strip comprises a polyamide that is electrostatically positively charged sufficiently to dislodge said tissue sample from said cradle by said touch-and-go motion.

15. The core biopsy tissue sample processing system of claim 10, in which said insert includes channels at sides of said strip platform that are open to each other and into which sides of said strip slide.

16. The core biopsy tissue sample processing system of claim 10, in which said insert includes surface features configured to retain said strip in place over said platform.

17. The core biopsy tissue processing system of claim 10, in which said insert is configured to fit in an industry-standard biopsy sample cassette that has an interior opening with sides 30-35 mm by 25-30 mm and 4-6 mm depth.

18. A core biopsy tissue sample processing system comprising:
a core biopsy needle that has a distal portion comprising an axially extending reduced-diameter portion configured to contain a tissue sample that has an exposed side; and
an insert comprising a strip platform, a strip of a material thereon that has a surface configured to dislodge a tissue sample from said reduced-diameter portion by lightly touching the exposed side of the tissue sample in a touch-and-go freehand motion of the biopsy needle relative to the strip, without a need for an adhesive.

19. The core biopsy tissue sample processing system of claim 18, in which the strip comprises Nylon 66 that is electrostatically charged positively sufficiently to dislodge said negatively tissue sample without adhesives in said touch-and-go motion.

20. The core biopsy tissue sample processing system of claim 18, in which said strip comprises Cellulose Chromatography Paper that is configured to dislodge said tissue sample without adhesives in said touch-and-go motion.

21. The core biopsy tissue sample processing system of claim 18, in which said insert is dimensioned to fit in an industry-standard biopsy sample cassette that has an interior opening with sides 30-35 mm by 25-30 mm and 4-6 mm depth.

22. A core biopsy tissue sample processing system comprising:
a core biopsy needle that has a distal portion with an elongated tissue sample receiving part that has reduced cross-sectional area compared with a more distal part of the needle;
an insert comprising an elongated tissue-receiving portion configured to receive a tissue sample from said biopsy needle; and
an industry-standard tissue biopsy sample cassette that has an interior opening with sides 30-35 mm and 25-30 mm long and has a 4-6 mm depth;
wherein said insert is sized and shaped to securely fit in said industry-standard cassette and has side portions that are adjacent to said sides of the cassette when inside the cassette and a height less than the depth of the cassette.

23. The core biopsy tissue processing system of claim 22, in which said insert has a first side that is 25 mm long and a second side that is 30 mm long and a height of 5 mm.

24. The core biopsy tissue processing system of claim 22, in which said insert has a first side that is no more than 25 mm long and a second side that is no more than 30 mm long.

25. A core biopsy tissue sample processing method comprising:
collecting a core biopsy sample with a core biopsy needle that has a distal portion with an elongated tissue sample receiving part that has reduced cross-sectional area compared with a more distal part of the needle;
dislodging a tissue sample from the needle into an insert comprising an elongated tissue-receiving portion configured to receive a tissue sample from said biopsy needle; and
placing the insert into an industry-standard tissue biopsy sample cassette that has an interior opening with sides 30-35 mm and 25-30 mm long and has a 4-6 mm depth such that the securely fits in said industry-standard cassette, with side portions if the insert adjacent to said sides of the cassette.

26. The core biopsy tissue processing method of claim 25, in which said placing of the insert into the cassette comprises placing an insert that has a first side that is 25 mm long and a second side that is 30 mm long and height of 5 mm.

27. The core biopsy tissue processing method of claim 25, in which said placing of the insert into the cassette comprises placing an insert that has a first side that is no more than 25 mm long and a second side that is no more than 30 mm long.
